(19) United States
(12) Patent Application Publication
(10) Pub. No.: US 2017/0252609 A1
(43) Pub. Date: Sep. 7, 2017

(54) SWIMMING GOGGLE CUSHION PAD

(71) Applicant: Global Esprit Inc., New Taipei City (TW)

(72) Inventor: HERMAN CHIANG, New Taipei City (TW)

(73) Assignee: Global Esprit Inc., New Taipei City (TW)

(21) Appl. No.: 15/083,132

(22) Filed: Mar. 28, 2016

(30) Foreign Application Priority Data
Mar. 4, 2016 (TW) 105203042

(51) Int. Cl. A63B 33/00; A61F 9/02

(52) U.S. Cl. CPC A63B 33/002; A61F 9/026; A63B 2033/004

(57) ABSTRACT

A swimming goggle cushion pad is connected to a frame of swimming goggles, wherein the cushion pad comprises a base, a face contact portion mounted on a side of the base and a lens frame disposed on another side of the base in opposite to the face contact portion, the lens frame being defined with an inner peripheral face and an outer peripheral face with a circumferential fitting groove arranged between the inner peripheral face and the outer peripheral face to hold a lens therein, characterized in that: the side of the base mounted with the face contact portion slightly bends inward along an outer peripheral contour of the base to form a cap shape, and the face contact portion comprises two arc surfaces of attachment to a wearer's face so as to provide complete coverage on the eye socket, regardless of various sizes of the wearer's face.

### BACKGROUND OF THE INVENTION

1. Field of the Invention

The present invention relates to a swimming goggle cushion pad, and particularly to a swimming goggle cushion pad capable of providing sufficient compliance with a wearer's face so as to provide a comfortable and soft engagement in wearing and provide a favorable effect in water leakage proof.

2. Related Art

Swimming goggles for protection of a wearer's eyes in swimming are designed in consideration of not only water leakage proof, but also wearing comfort. Conventionally, the swimming goggles cushion pad in contact with a wearer's face has two types. The first one is made of foam material and the second one comprises a suction cup like configuration. The foam cushion pad of the first type is attached to the wearer's face skin around the eye socket for water leakage proof by means of its material property of flexibility. In general, the wearer's face skin around the eye socket has an irregular contour in relation to each swimming goggle cushion pad allocating at least two different thicknesses corresponding to the eyebrow and the zygomatic bone in order to preferably conform to the irregular contour of the wearer's face skin around the eye socket. Unfortunately, the overall thickness of the foam cushion pad appears to be unitary, that is, when worn on the wearer's head, the cushion pad needs to be hardly pressed against the wearer's face skin around the eye sockets in requirement of a desired water tight engagement.

The suction cup like cushion pad of the second type usually has a J-shaped cross section which allows the cushion pad to collapse upon contact with the wearer's face skin around the eye socket. Since the J-shaped cushion pad has same upper and lower half arc contours and same upper and lower half cross sectional widths, the cushion pad needs to be pressed hardly against the wearer's face skin around the eye socket in requirement of the desired water tight engagement as well. Since the cushion pad, either of the foam type or the suction cup type, provides the desired water tight engagement with the wearer's face by being pressed hardly against the wearer's face skin around the eye socket, which may provide a persistent pressure inside the goggles, the pressure condition inside the goggles causes stress on the eye socket and thus the panda eye comes out in taking off the goggles, which also indicates an uncomfortable contact between the cushion pad and the wearer's face.

Furthermore, for wearers having different face sizes or shapes, the suction cup like cushion pad is more likely to cause water leakage. An example of conventional swimming goggles comprising the suction cup like cushion pad is shown in FIGS. 1 and 2. As shown, the suction cup like cushion pad 5 mounted on a lens frame 6 is to cover the wearer's face skin around the eye socket. The suction cup like cushion pad 5 has a base 50 and a face contact portion 51. As seen from the contour, the base 50 extends backward to form an open end. The face contact portion 51 is arranged around the open end of the base 50 and has same upper half contour and lower half contour. For such type worn by a wearer whose face size is small, the suction cup like cushion pad 5 provides poor coverage on the eye socket in lack of sufficient compliance with the wearer's face skin around the eye socket, thereby increasing risks in water leakage. When worn by a wearer whose face size is relatively larger, the suction cup like cushion pad 5 is easily detached from the wearer's face and curls up at its outer edges, as represented by dotted lines in FIG. 1, while a head strap 52 is pulled outward, as represented by arrow symbols in FIG. 1, thereby increasing risks in water leakage as well. Briefly, the conventional suction cup like cushion pad with the same upper and lower half arc contours constructed fails to provide sufficient compliance with wearer's face contour so that it only creates a very poor water leakage proof attachment to the wearer's face.

### SUMMARY OF THE INVENTION

An object of the present invention is to provide a swimming goggle cushion pad, which provides an engagement with a wearer's face skin around the eye socket and provides complete coverage on the eye socket, and thus the water leakage may be eliminated.

Another object of the present invention is to provide a swimming goggle cushion pad, which provides sufficient compliance with the wearer's face so as to provide a comfortable and soft engagement in wearing and cause no stress on the eye sockets.

To attain this, the swimming goggle cushion pad is connected with swimming goggles, wherein the cushion pad comprises a base, a face contact portion mounted on a side of the base, characterized in that: the side of the base mounted with the face contact portion slightly bends inward along an outer peripheral contour of the base to form a cap shape for strengthening the attachment of the face contact portion to a wearer's face , and further, the face contact portion comprises a first arc surface in contact with the wearer's face skin around the inner eye corner and a second arc surface in contact with the wearer's face skin around the outer eye corner, wherein the first and second arc surfaces connect with each other at a minimum angle to form a plane-like surface to ensure a smooth engagement with the wearer's face skin around the eye socket.

In accordance with the present invention, the cushion pad further comprises a lens frame which is disposed on another side of the base in opposite to the face contact portion, and defined with an inner peripheral face and an outer peripheral face, wherein a circumferential fitting groove is arranged between the inner peripheral face and the outer peripheral face to hold lens therein, and wherein the outer peripheral face of the lens frame is punched inward at intervals to form a plurality of clamping grooves to which a frame of the swimming goggle is fixed.

In accordance with the present invention, wherein a thickness of the cushion pad from the base to the face contact portion becomes thinner and thinner, so as to provide a thicker base for supporting and a thinner face contact portion for comfortable engagement with the wearer's face skin around the eye socket.

In accordance with the present invention, wherein the cushion pad has an upper half part with respect to the wearer's eyebrow having a first height measured from the base to the face contact portion, and a lower half part with respect to the wearer's zygomatic bone having a second height measured from the base to the face contact portion, wherein the first height is made smaller than the second height, so that the cushion pad is able to comply with the wearer's skull upon the collapse of the base without causing stress on the eye socket.

### BRIEF DESCRIPTION OF THE DRAWINGS

FIG. 1 is a top view showing a pair of conventional swimming goggles of which the cushion pad has a suction cup like configuration;

FIG. 2 is a top view showing the cushion pad of the conventional swimming goggles having the suction cup like configuration;

FIG. 3 is a perspective view showing a swimming goggle cushion pad in accordance with the present invention;

FIGS. 4A and 4B and 5 are a top view and a bottom view and a front view of FIG. 3;

FIG. 5A is a cross-sectional view taken along line A-A of FIG. 5;

FIG. 5B is a cross-sectional view taken along line B-B of FIG. 5;

FIG. 5C is a cross-sectional view taken along line C-C of FIG. 5;

FIGS. 6 and 7 are an exploded perspective view and an assembled perspective view showing the swimming goggle cushion pad in accordance with the present invention connected to the lens and the frame of the swimming goggles;

FIGS. 8A and 8B are top views showing the left and right swimming goggle cushion pads in accordance with the present invention in comparison with the conventional left and right swimming goggle cushion pads;

FIGS. 9A and 9B are schematic views showing the swimming goggle cushion pad in accordance with the present invention in comparison with the conventional swimming goggle cushion pad.

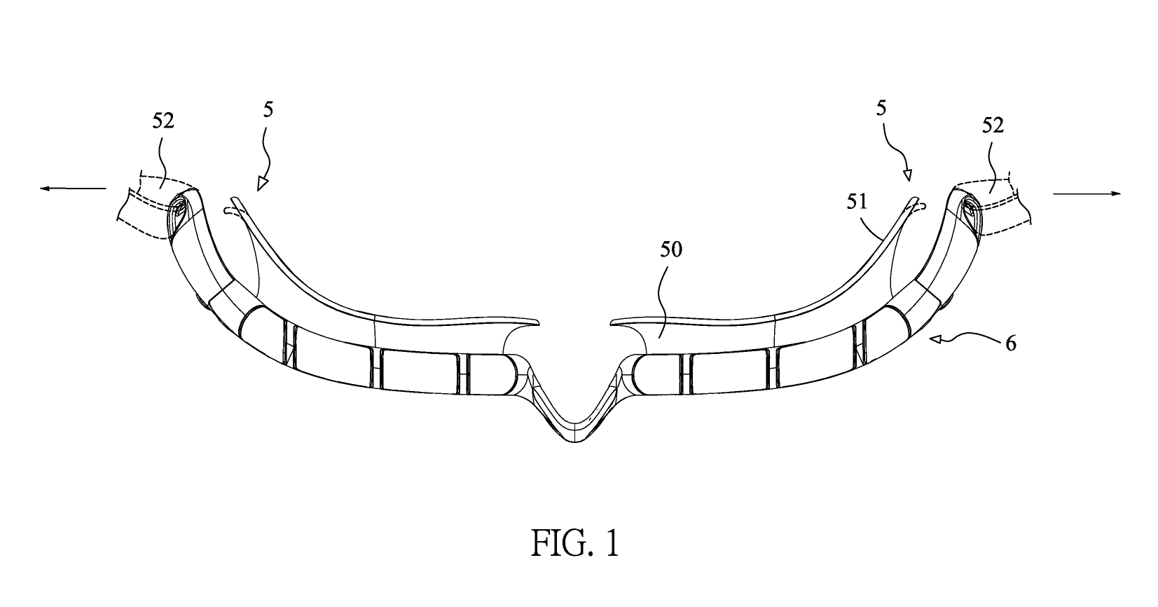

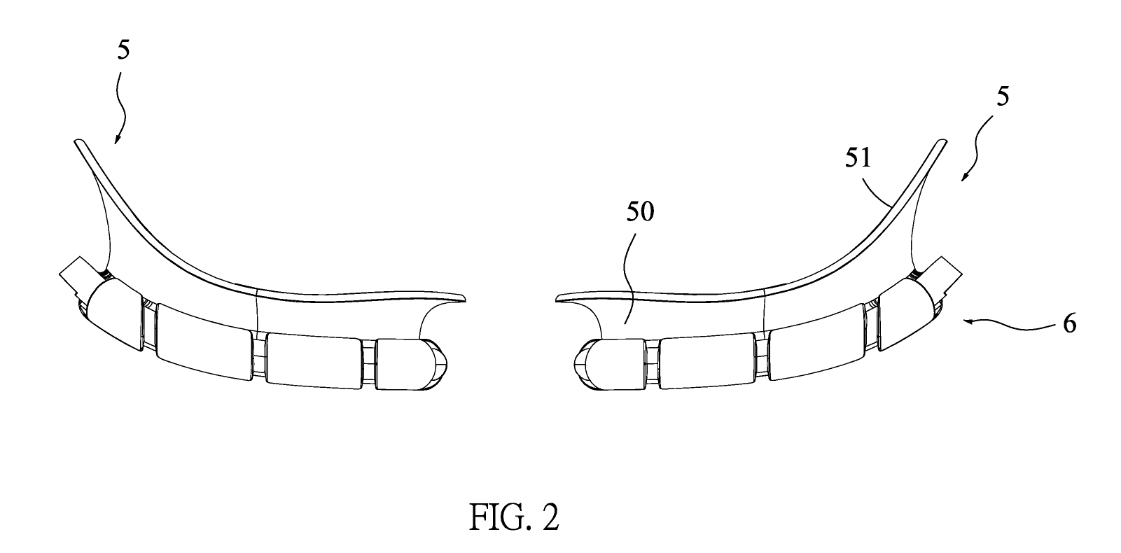

### DESCRIPTION OF THE PREFERRED EMBODIMENTS

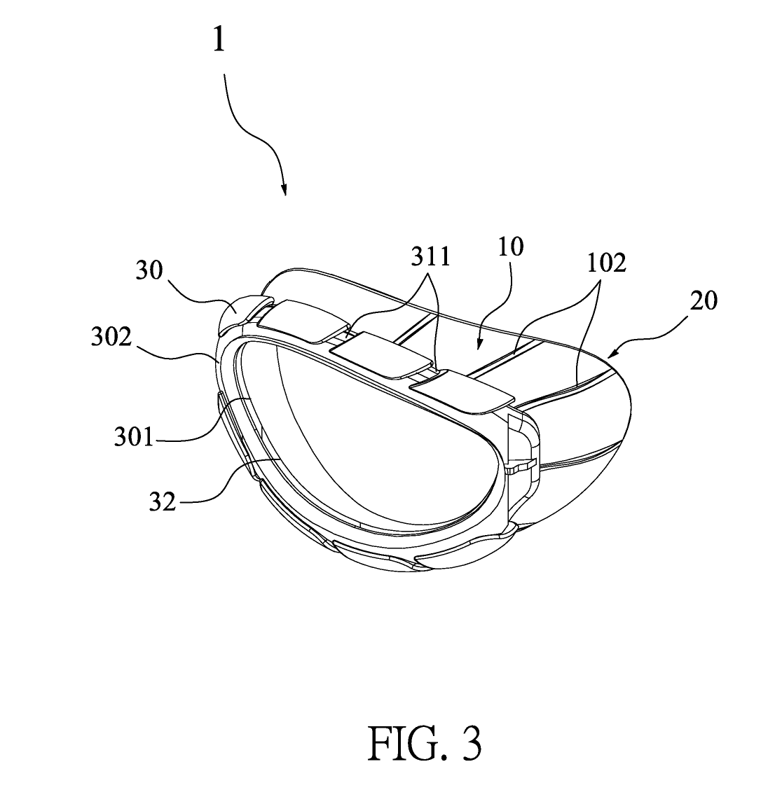

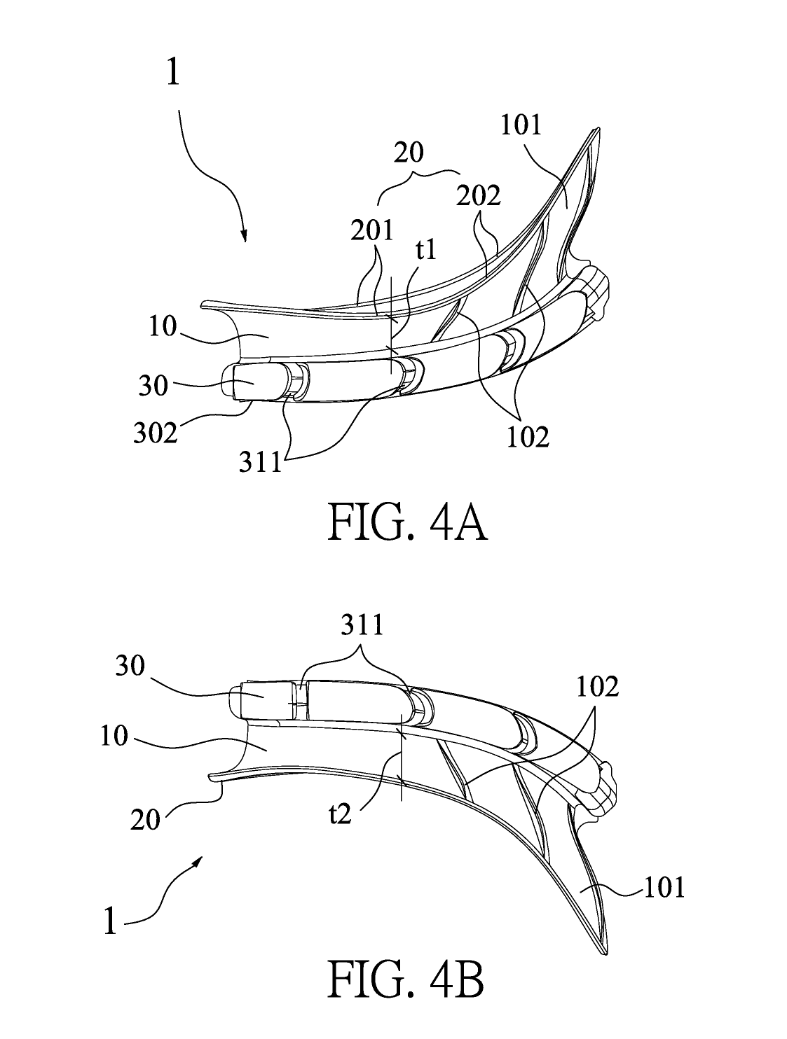

Referring to FIGS. 3, 4A and 4B, each cushion pad 1 for a pair of swimming goggles in accordance with the present invention is shown. The cushion pad 1 comprises a base 10, a face contact portion 20 and a lens frame 30. The face contact portion 20 is mounted on a side of the base 10. The lens frame 30 is disposed on another side of the base 10 in opposite to the face contact portion 20. The lens frame 30 made of Thermo Plastic Rubber (TPR) or silicon material is defined with an inner peripheral face 301 and an outer peripheral face 302. A circumferential fitting groove 32 is arranged between the inner peripheral face 301 and the outer peripheral face 302 of the lens frame 30 for receiving and holding each lens therein. The base 10 may be of the same material as the lens frame 30 and integrally formed with the lens frame 30. The side of the base 10 mounted with the face contact portion 20 slightly bends inward along an outer peripheral contour of the base 10 to form a cap shape 101 for strengthening the attachment of the face contact portion 20 to a wearer's face, so that the cushion pad 1 in accordance with the present invention provides complete coverage on the wearer's eye socket, regardless of various sizes of the wearer's face. A plurality of reinforcing ribs 102 are disposed along the outer peripheral contour of the base 10 and interconnects the face contact portion 20 and the lens frame 30 to support the cap shape 101.

Further, the face contact portion 20 disposed in opposite to the lens frame 30 and integrally formed with the base 10, comprises two arc surfaces 201,202 of attachment to the wearer's face on which a first arc surface 201 in contact with the wearer's face skin around the inner eye corner and a second arc surface 202 in contact with the wearer's face skin around the outer eye corner, wherein the first and second arc surfaces 201,202 connect with each other at a minimum angle to form a plane-like surface to ensure a smooth engagement with the wearer's face skin around the eye socket. Further, the outer peripheral face 302 of the lens frame 30 is punched inward at intervals to form a plurality of clamping grooves 311 to which swimming goggle components including a clamping frame formed with a head strap can be fixed. In practice, whether clamping grooves 311 is mounted on the outer peripheral face 302 of the lens frame 30 depends on the configuration of the clamping frame. The lens frame 30 having clamping grooves 311 thereon is for use in swimming goggles having clamping frames. For swimming goggles equipped with no clamping frames, the lens frame 30 may have one single or two separate head straps disposed on both sides thereof and may be integrally formed with the base 10 and the face contact portion 20 of the cushion pad 1.

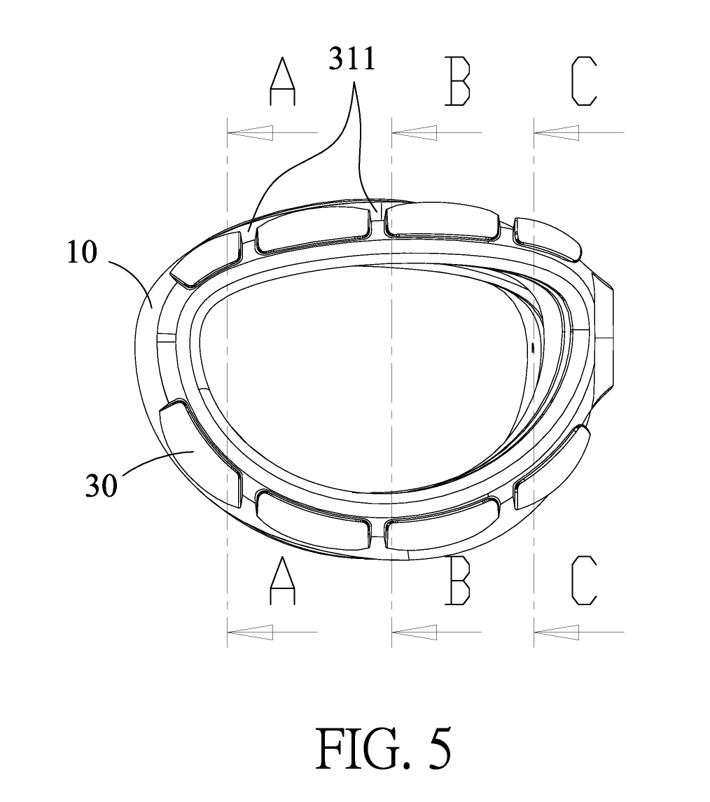

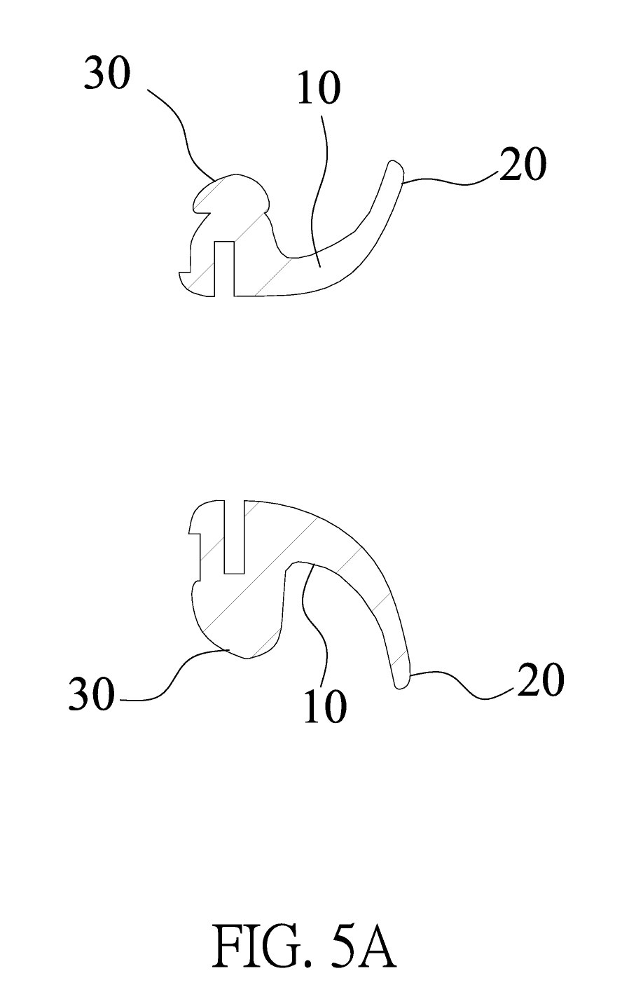

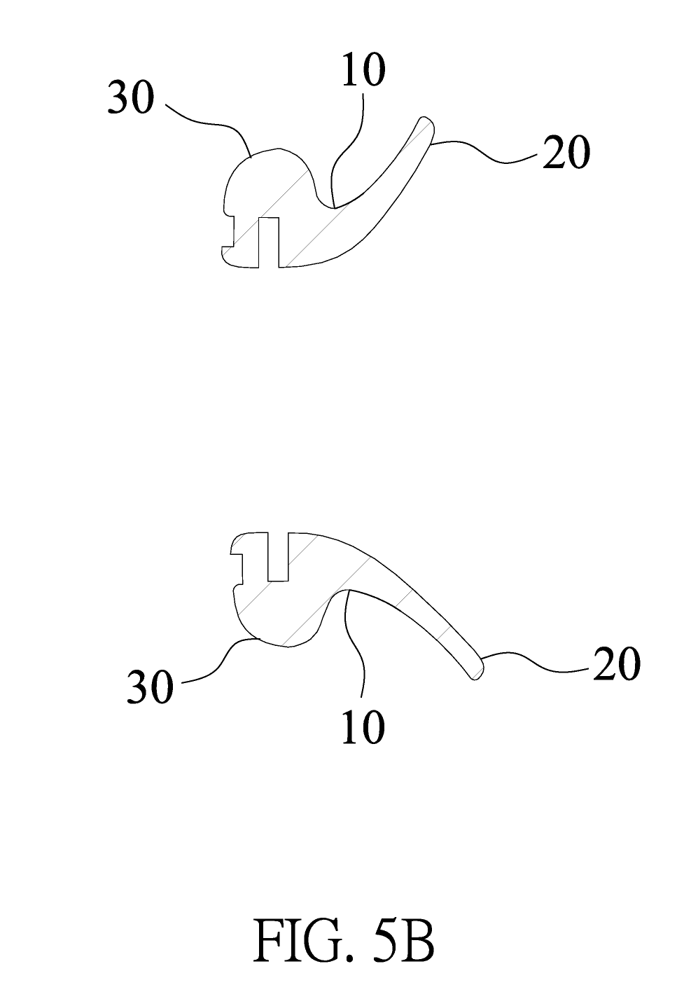

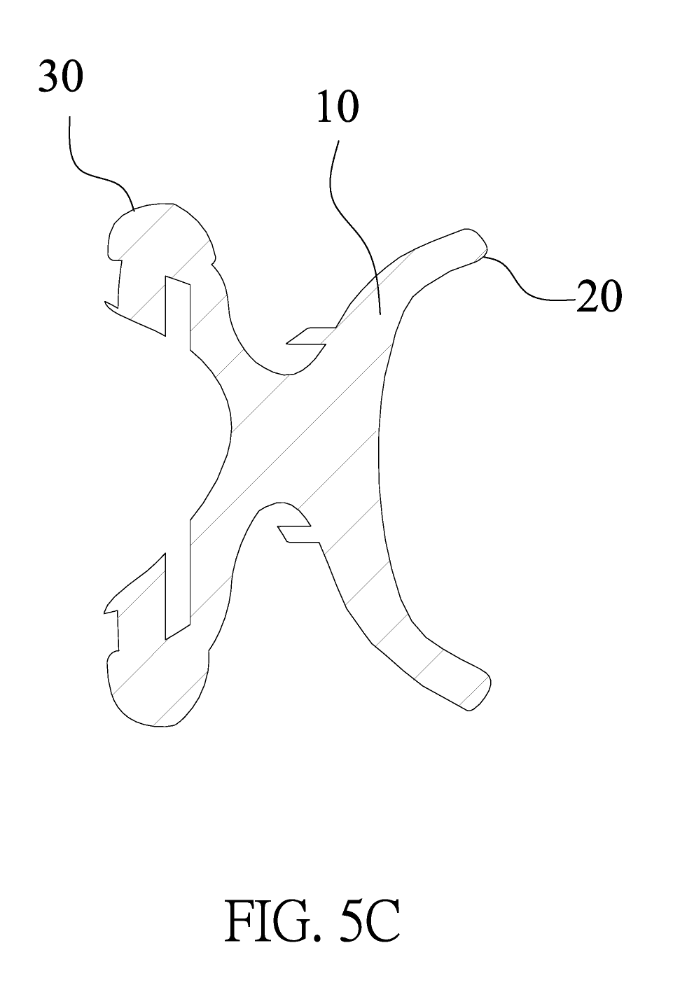

Referring to FIGS. 5, 5A, 5B and 5C, a thickness of the cushion pad 1 from the base 10 to the face contact portion 20 becomes thinner and thinner, so as to provide a thicker base 10 for supporting and a thinner face contact portion 20 for comfortable engagement with the wearer's face skin around the eye socket. With reference to FIGS. 4A and 4B, the cushion pad 1 is composed of an upper half part with respect to the wearer's eyebrow having a first height t1 (FIG. 4A) measured from the base 10 to the face contact 20, and a lower half part with respect to the wearer's zygomatic bone having a second height t2 (FIG. 4B) measured from the base 10 to the face contact 20, wherein the first height t1 is made smaller than the second height t2, so that the cushion pad 1 having different heights on upper and lower half parts thereof is able to comply with the wearer's skull upon the collapse of the base 10 without causing stress on the eye socket.

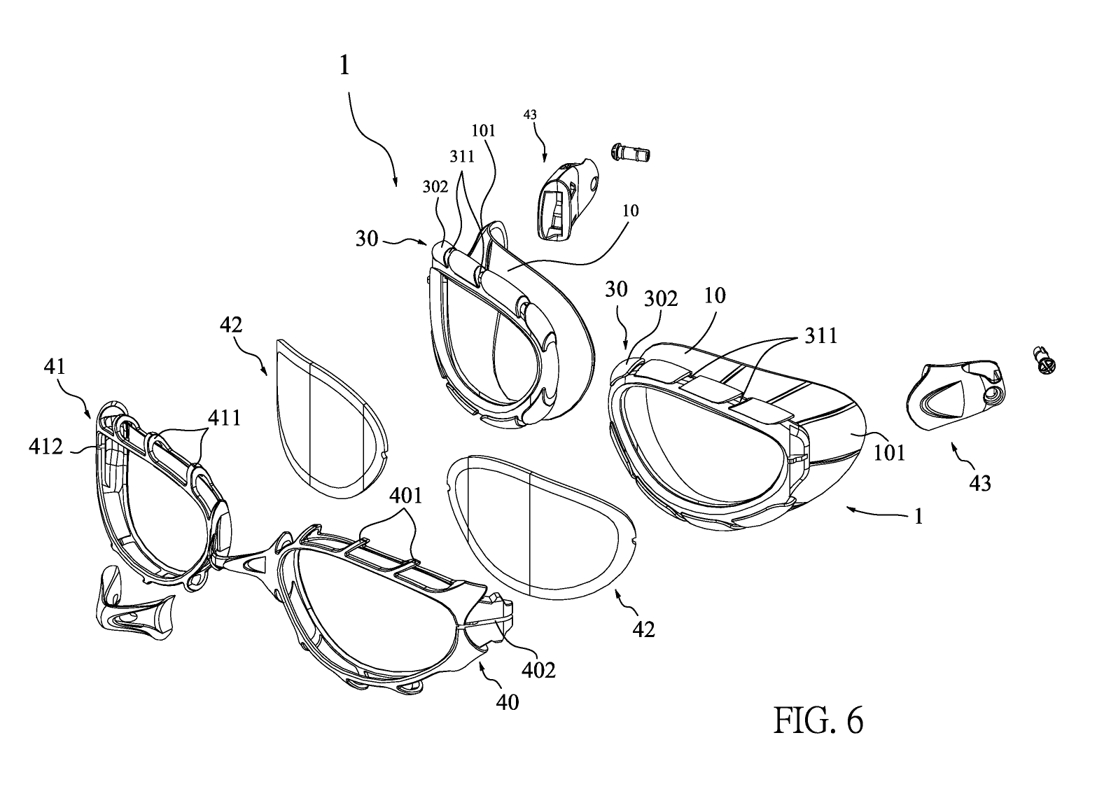

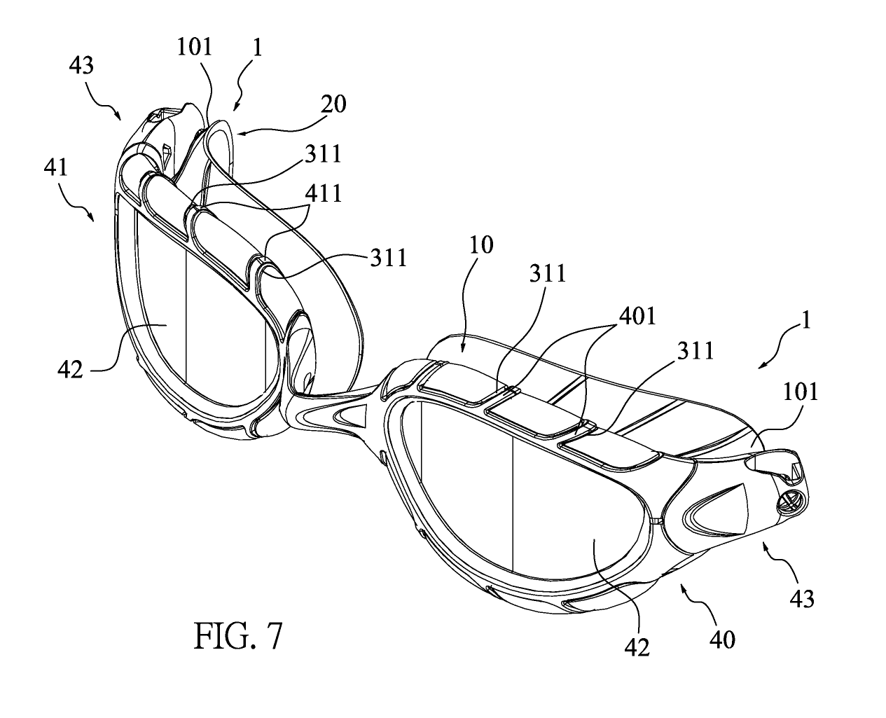

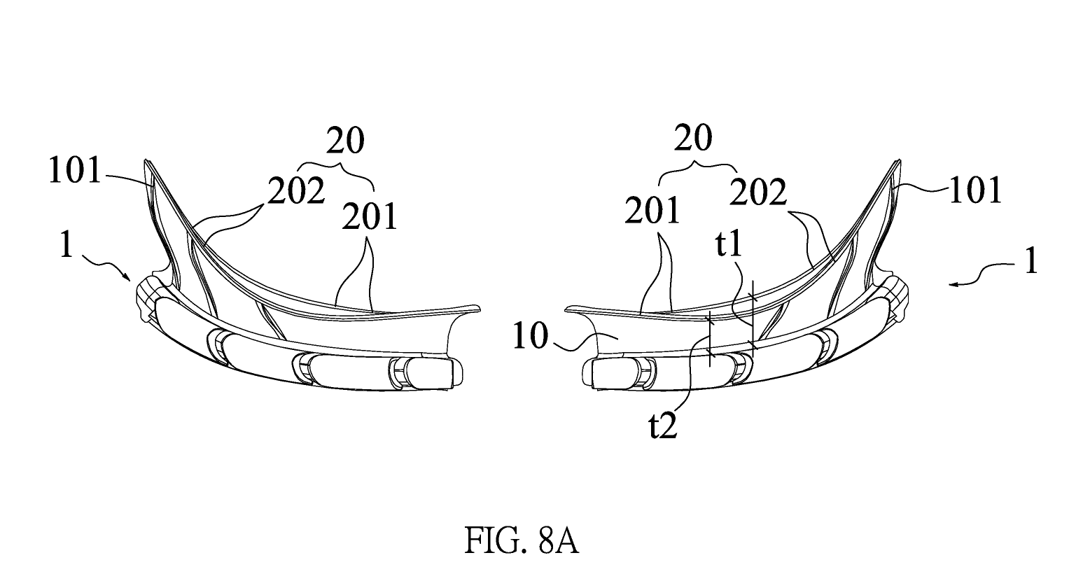

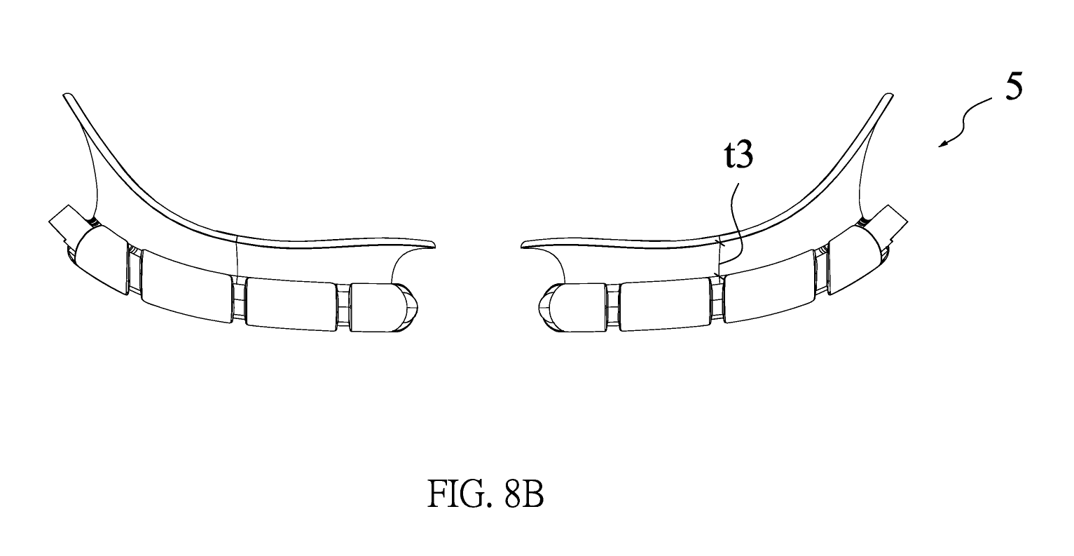

Referring to FIG. 6, the cushion pad 1 is incorporated in a pair of swimming goggles in an embedding manner such that left and right clamping frames 40,41 respectively have a plurality of embedding elements 401, 411 capable of being embedded in the clamping grooves 311 of the outer peripheral face 302 of the lens frame 30. In assembly, after the lens is positioned in the circumferential fitting groove 32, the embedding elements 401, 411 are embedded in the clamping grooves 311, and packing elements 43 attached with a head strap (not shown) are embedded into slots 402, 412 of the left and right clamping frames 40,41, so that the cushion pads 1 are fixed to the left and right clamping frames 40,41, as shown in FIG. 7. Referring to FIG. 8A, 8B, 9A and 9B, illustrating the cushion pads 1 in accordance with the present invention compared with conventional cushion pads 5, there are several differences, as follows:

First, the cushion pad 1 in accordance with the present invention comprises the base 10 having the cap shape 101 (FIG. 8A) bending inward along the outer peripheral contour thereof for strengthening the attachment of the face contact portion 20 to the wearer's face, and the two arc surfaces 201,202 in contact with the wearer's face skin around the inner and outer eye corners so as to provide complete coverage on the wearer's face skin around the eye socket without water leakage. In contrast, the conventional cushion pad 5 is defined only with a single arc surface with no cap shape formed by bending inward along an outer peripheral contour of its base (FIG. 8B), which provides weak coverage on the wearer's face skin around the eye socket and may cause water leakage.

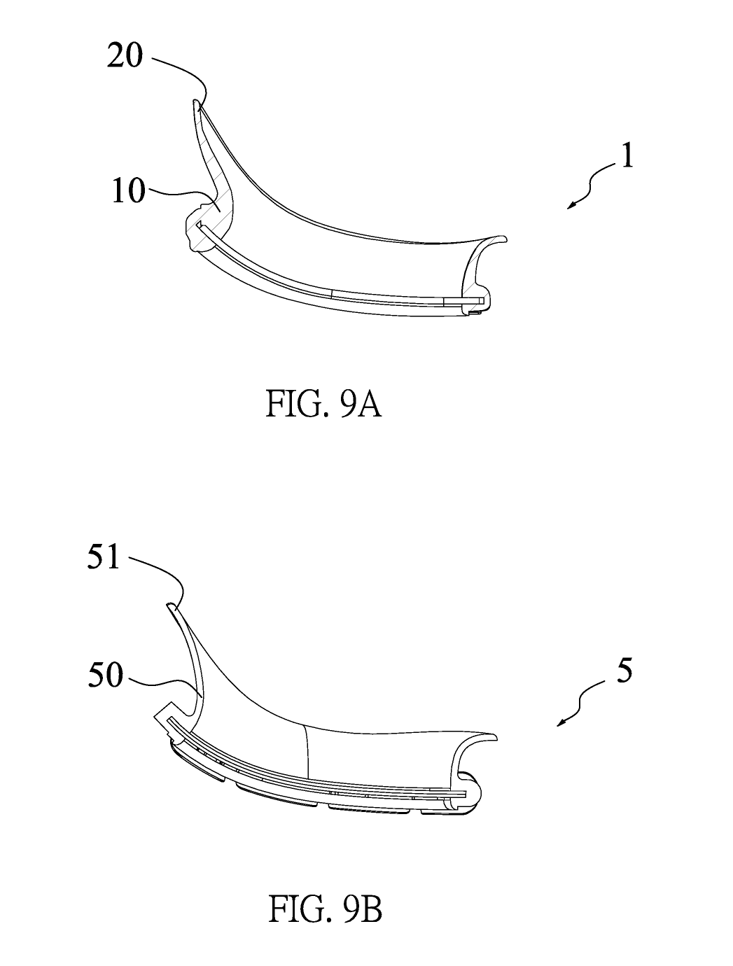

Second, the thickness of the cushion pad 1 from the base 10 to the face contact portion 20 becomes thinner and thinner (FIG. 9A), so as to provide a thicker base 10 for supporting and a thinner face contact portion 20 for comfortable engagement with the wearer's face skin around the eye socket. However, the conventional cushion pad 5 is of a unitary thickness throughout its base 50 and face contact portion 51 (FIG. 9B) so that it could not reach a comfortable engagement with face skin.

Third, the upper half part of the cushion pad 1 with respect to the wearer's eyebrow has a first height t1 (FIG. 8A) measured from the base 10 to the face contact portion 20, and the lower half part of the cushion pad 1 with respect to the wearer's zygomatic bone has a second height t2 (FIG. 8A) measured from the base 10 to the face contact portion 20, wherein the first height t1 is made smaller than the second height t2, so that the cushion pad 1 having different heights on upper and lower half parts thereof can comply with the wearer's skull upon the collapse of the base 10 without causing stress on the eye socket. In contrast, the upper and lower half parts of the conventional cushion pad 5 with respect to the wearer's eyebrow and zygomatic bone both have a same height t3 (FIG. 8B), so that it could not comply with the wearer's skull of which the eyebrow and zygomatic bone have non-uniform depths, so as not to provide a wearing comfort.

It is understood that the invention may be embodied in other forms within the scope of the claims. Thus the present examples and embodiments are to be considered in all respects as illustrative, and not restrictive, of the invention defined by the claims.

What is claimed is:

1. A swimming goggle cushion pad, connected to a frame of swimming goggles, wherein the cushion pad comprises a base, a face contact portion mounted on a side of the base and a lens frame disposed on another side of the base in opposite to the face contact portion, the lens frame being defined with an inner peripheral face and an outer peripheral face with a circumferential fitting groove arranged between the inner peripheral face and the outer peripheral face to hold a lens therein, characterized in that: the side of the base mounted with the face contact portion slightly bends inward along an outer peripheral contour of the base to form a cap shape, and the face contact portion comprises two arc surfaces of attachment to a wearer's face on which a first arc surface in contact with the wearer's face skin around the inner eye corner and a second arc surface in contact with the wearer's face skin around the outer eye corner.

2. The swimming goggle cushion pad of claim 1, wherein a thickness of the cushion pad from the base to the face contact portion becomes thinner and thinner, so as to provide a thicker base for supporting and a thinner face contact portion for comfortable engagement with the wearer's face skin around the eye socket.

3. The swimming goggle cushion pad of claim 1, wherein the cushion pad is composed of an upper half part with respect to the wearer's eyebrow having a first height measured from the base to the face contact portion, and a lower half part with respect to the wearer's zygomatic bone having a second height measured from the base to the face contact portion, wherein the first height is made smaller than the second height, so that the cushion pad having different heights on the upper and lower half parts thereof complies with the wearer's skull upon the collapse of the base without causing stress on the eye socket.

4. The swimming goggle cushion pad of claim 1, wherein a plurality of reinforcing ribs are disposed along the outer peripheral contour of the base and interconnects the face contact portion and the lens frame to support the cap shape.

5. The swimming goggle cushion pad of claim 1, wherein the outer peripheral face of the lens frame is punched inward at intervals to form a plurality of clamping grooves to which the frame of the swimming goggle is fixed.

6. The swimming goggle cushion pad of claim 1, wherein the first and second arc surfaces connect with each other at a minimum angle to form a plane-like surface to ensure a smooth engagement with the wearer's face skin around the eye socket.

7. A swimming goggle cushion pad, integrally formed with a frame of swimming goggles, wherein the cushion pad comprises a base, a face contact portion mounted on a side of the base, characterized in that: the side of the base mounted with the face contact portion slightly bends inward along an outer peripheral contour of the base to form a cap shape, and the face contact portion comprises two arc surfaces of attachment to a wearer's face on which a first arc surface in contact with the wearer's face skin around the inner eye corner and a second arc surface in contact with the wearer's face skin around the outer eye corner.

8. The swimming goggle cushion pad of claim 7, wherein a thickness of the cushion pad from the base to the face contact portion becomes thinner and thinner, so as to provide a thicker base for supporting and a thinner face contact portion for comfortable engagement with the wearer's face skin around the eye socket.

9. The swimming goggle cushion pad of claim 7, wherein a plurality of reinforcing ribs are disposed along the outer peripheral contour of the base and interconnects the face contact portion and the lens frame to support the cap shape.

10. The swimming goggle cushion pad of claim 7, wherein the first and second arc surfaces connect with each other at a minimum angle to form a plane-like surface to ensure a smooth engagement with the wearer's face skin around the eye socket.
